(19) United States
(12) Patent Application Publication
(10) Pub. No.: US 2026/0007859 A1
(43) Pub. Date: Jan. 8, 2026

(54) SUTURELESS DRAIN SECUREMENT DEVICE

(71) Applicant: Northwestern University, Evanston, IL (US)

(72) Inventors: Riad SALEM, Evanston, IL (US); Abhinav TALWAR, Evanston, IL (US); Ahsun RIAZ, Evanston, IL (US)

(21) Appl. No.: 18/993,458

(22) PCT Filed: Jul. 13, 2023
(86) PCT No.: PCT/US23/70142

§ 371 Date: Jan. 10, 2025

Related U.S. Application Data
(60) Provisional application No. 63/388,916, filed on Jul. 13, 2022.

(51) Int. Cl. A61M 25/02

(52) U.S. Cl. CPC A61M 25/02; A61M 2025/024; A61M 2025/0266

(57) ABSTRACT

Provided herein are devices for sutureless securement of medical devices to a subject. In particular, drain tubes (e.g., percutaneous drains), catheters, and other medical devices are secured to the skin of a patient (e.g., near the insertion site) using the devices herein.

### CROSS-REFERENCE TO RELATED APPLICATIONS

This application claims the benefit of U.S. Provisional Patent Application No. 63/388,916, filed on Jul. 13, 2022, which is incorporated by reference herein.

### FIELD

Provided herein are devices for sutureless securement of medical devices to a subject. In particular, drain tubes (e.g., percutaneous drains), catheters, and other medical devices are secured to the skin of a patient (e.g., near the insertion site) using the devices herein.

### BACKGROUND

Clinicians, for example, interventional radiologists (IR) and surgeons, place drains and other catheters percutaneously as conduits for fluid to be removed from bodily cavities. A growing body of evidence indicates that complications of drain placement—pain, leakage, dislodgment, and infection—occur frequently. Many of these complications are attributable, at least in part, to the methods used to place and secure drains. Suture is often used to tie drains to skin, but loose knots predispose to drain dislodgment while tight knots frequently cause pain and serve as niduses for infection. Adhesive drain fixation devices lose efficacy over time and make dressing changes unnecessarily complicated. What is needed is a device that allows for stable and secure attachment of drain/catheter devices, while allowing for patient movement and dressing changes, without producing discomfort of adverse events.

### SUMMARY

Provided herein are devices for sutureless securement of medical devices to a subject. In particular, drain tubes (e.g., percutaneous drains), catheters, and other medical devices are secured to the skin of a patient (e.g., near the insertion site) using the devices herein.

In some embodiments, provided herein are line securement devices comprising: (a) a securement element comprising: (i) a housing having an interior channel that traverses a vertical axis of the securement element, (ii) a channel that traverses a vertical axis of the securement element though the center of the housing and having openings on either end of the channel, wherein the channel is configured allow a line to extend through the center of the housing, along the vertical axis, and exiting the housing through the openings, and (iii) a hanger extending from the housing along a horizontal axis; (b) a receiving element comprising: (i) a mounting plate having a front face and a rear face, (ii) a receptacle attached to and extending from the front face of the mounting plate, wherein the receptacle is configured to receive the hanger, (iii) an adhesive element on the rear face of the mounting plate, wherein the adhesive element is configured to securely couple the mounting plate to the skin of a subject.

In some embodiments, the housing comprises a clam-shell having a rear body and a front body. In some embodiments, the front body is hingedly coupled to the rear body. In some embodiments, the housing comprises one or more clips for coupling the front body to the rear body (in place of or for use with a hinge). In some embodiments, the channel resides between the front body and the rear body. In some embodiments, the hanger extends from the rear body of the housing. In some embodiments, the hanger comprises one or more (e.g., 1, 2, etc.) rods (or pins or equivalents thereof) extending from the housing.

In some embodiments, the receptacle is a hook-shaped protrusion configured (e.g., sized and shaped) to partially (e.g., 330°, 315°, 300°, 270°, 255°, 240°, 225°, or ranges therebetween) circumscribe the cross-sectional shape of the rod. In some embodiments, the rod has a length and a cross-sectional diameter. In some embodiments, the rod is terminated by an end cap of greater diameter (and circumference) than the rod. In some embodiments, the hanger comprises a pair of rods located on opposite sides of the housing. In some embodiments, the receptacle comprises a pair of receptacles located on opposite sides of the mounting plate. In some embodiments, upon receipt of the hanger (e.g., pair of rods) by the receptacle, the securement element is securely coupled to the mounting element. In some embodiments, the receptacle is large enough to receive the rod but not the end cap. In some embodiments, the end caps is aligned outside of the receptacle when the receptacle receives the rod. In some embodiments, receipt of the hanger by the receptacle allows for the securement element to pivot (e.g., 15°, 30°, 45°, 60°, 75°, 90°, 105°, 120°, 135°, 150°, 165°, 180°, or ranges therebetween) in a single plane with respect to the mounting element.

In some embodiments, the channel is 5-100 Fr in diameter (e.g., 5 Fr, 10 Fr, 15 Fr, 20 Fr, 25 Fr, 30 Fr, 35 Fr, 40 Fr, 45 Fr, 50 Fr, 55 Fr, 60 Fr, 70 Fr, 80 Fr, 90 Fr, 100 Fr, or ranges therebetween (e.g., 10-30 Fr, etc.)).

In some embodiments, the adhesive element is a bandage attached to the rear face of the mounting plate. In some embodiments, the adhesive element is a glue applied to the rear face of the mounting plate.

In some embodiments, provided herein are systems comprising a line securement device described herein and a medical line. In some embodiments, the medical line is a percutaneous drain or a catheter. In some embodiments, the medical line extends through the channel. In some embodiments, the securement element is securely coupled to the receiving element by the hanger and receptacle.

In some embodiments, provided herein are methods of securing a medical line to a subject using a line securement device described herein, the method comprising: (a) placing the medical line at a first location on the subject; (b) running the medical line through the channel of the housing of the securement element; (c) adhering the receiving element to the subject by the adhesive element at a second location adjacent to the first location; and (d) coupling the securement element to the receiving element by the hanger and receptacle. In some embodiments, placing the medical line at a first location on the subject comprises inserting the medical line into an incision in the subject or an orifice of the subject. In some embodiments, the medical line is a percutaneous drain or a catheter. In some embodiments, running the medical line through the channel comprises aligning the rear and front bodies of the housing with the medical line and coupling the front body to the rear body, thereby securing the medical line within the channel of the housing.

### BRIEF DESCRIPTION OF THE DRAWINGS

FIG. 1A-B. Views of a securement element 10 according to one embodiment. (A) Perspective views of a securement element 10: left—both the rear 12 and front 13 bodies of the housing 11; right—rear body 12 of the housing 11, revealing the channel 14 traversing the housing along a central vertical axis 40. (B) Front (left), side (middle), and top (right) views of a securement element 10 according to one embodiment; clips 15 for securing the front body 13 to the rear body 12 of the clam-shell housing 11 and rods 16 positioned along a horizontal axis 50 for attachment to the mounting plate (not pictured in FIG. 1) are depicted.

FIG. 2A-B. Views of a receiving element 20 according to one embodiment. (A) Perspective (far left), front (middle left), side (middle right), and top (far right) views of a receiving element 20, depicting mounting plate 21 and receptacles 22. (B) front view of a receiving element 20 in which the mounting plate 21 is attached to an exemplary adhesive element 23.

FIG. 3. Views of a device 1 according to one embodiment. The securement element 10 is pivotally engages with the receiving element 20.

FIG. 4. Drain securement system according to one embodiment. (1) Percutaneous drain and incisions site in the skin. (2) Rear body of securement element housing with drain line residing in the vertical channel. (3) Drain line traversing the vertical channel within the complete housing, with the front body closed onto the rear body. (4) Dressing (adhesive element) placed over the incision site and presenting the receiving element, allowing the drain to pass through the bandage. (5) Securement element on drain engaged with receiving element adhered to skin via the adhesive element.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

### DETAILED DESCRIPTION

Provided herein are devices for sutureless securement of medical devices to a subject. In particular, drain tubes (e.g., percutaneous drains), catheters, and other medical devices are secured to the skin of a patient (e.g., near the insertion site) using the devices herein.

Provided herein are medical devices that function to fix a medical line (e.g., catheter, percutaneous drain, IV tubing, etc.) to a patient without the use of a suture. In some embodiments, the devices herein allow the medical line to be stably held in place with respect to the point of entry into the subject (e.g., an incision or orifice). In some embodiments, a device allows the patient to move while the line is secured by the device (e.g., without disrupting the insertion of the line). In some embodiments, a device allows the position of the body of the medical line (not the insertion stie) to be adjusted while the line is secured by the device. Certain embodiments are described herein for use with percutaneous drains; however, the embodiments described herein may also find use in the sutureless securement of other medical lines, such as catheters (e.g., urinary catheters) and IV tubing. Embodiments herein are not limited by the identity of the medical lines to be used therewith.

In some embodiments, the devices herein are sutureless drain fixation devices that are more comfortable for patients, easier to exchange during dressing changes, and decrease the incidence of drain-related adverse events. In some embodiments, the drain securement devices herein do not evert or tug at skin which provides increased comfort for patients. In some embodiments, securement devices herein apply circumferential pressure around the site of insertion (e.g., drain) to provide strong fixation, decreased risk of dislodgment, and decreased risk of kinking/obstruction. In some embodiments, because the tip of the drain can move freely in one plane, the devices herein provide decreased intra-corporeal “play” (i.e., the end of the drain in the body moves less; is more stable) when the outside tip is manipulated. In some embodiments, devices herein decrease the risk of dislodgment compared to existing techniques. In some embodiments, because the tubing does not adhere to the device, the device allows for quicker and easier changing of the drain, dressing, etc. In some embodiments, the incision site is not exposed to suture which can predispose to infection. Therefore, the devices herein decrease the risk of infection.

In particular embodiments, provided herein are devices for securing a medical line (e.g., tube) at an incision site of a patient. In some embodiments, the medical line is a percutaneous drain. Exemplary medical lines that find use with embodiments of the devices herein may include an endotracheal tube, a tracheotomy tube, chest tube, a vascular catheter, an urinary catheter, a nephrostomy tube, a biliary stent, a peritoneal catheter, an epidural catheter, a central nervous system catheter, a prosthetic valve, a gastric tube, an intestinal tube, drug-delivery implant, or an intravenous tube. In some embodiments, the devices herein find use in securing any medical line that enters the subject (e.g., at an incision or through an orifice) and needs to be held in place relative to the entry site and/or on the skin of the subject.

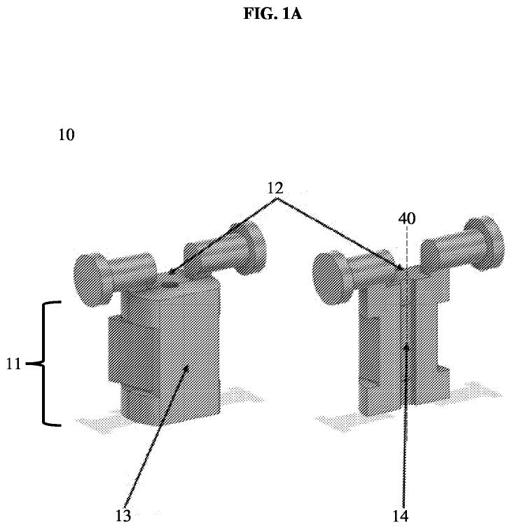

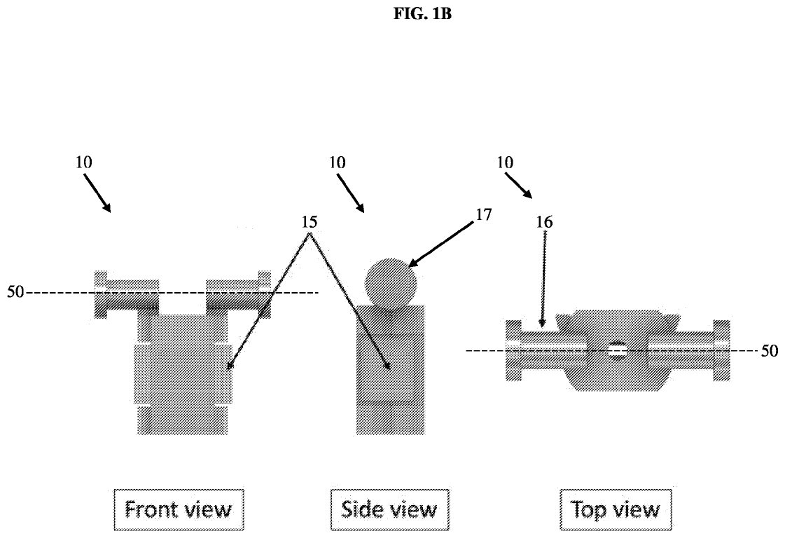

In some embodiments, the devices herein comprise a securement element. The securement element engages with the medical line. In some embodiments, the medical line resides within a channel through the securement element. A non-limiting example of a securement element that finds use in certain embodiments herein is depicted in FIG. 1. As depicted in FIG. 1A, in some embodiments, a securement element 10 comprises a housing 11 having a rear body 12 and front body 13. In some embodiments, and interior channel 14 traverses the housing along a central vertical axis 40. As depicted in FIG. 1B, in some embodiments, a securement element 10 comprises one or more clips 15 for securing the front body 13 to the rear body 12 of the clam-shell housing 11 and rods 16 positioned along a horizontal axis 50 for attachment to the mounting plate.

In some embodiments, the medical line is inserted through the channel within the securement element. In some embodiments, the housing of the securement element is assembled around the medical line, thereby enclosing a portion of the medical line within the securement element. In some embodiments, the housing of the securement element is closed upon the medical line, thereby enclosing a portion of the medical line within the securement element.

The housing may include a housing and a vertical channel about which the body is located, the housing being adapted to allow the line (e.g., drain, catheter, etc.) to reside within the vertical channel when the housing is engaged with the line (e.g., drain, catheter, tube, etc.). In some embodiments, the housing is configured to allow portions of the line (e.g., drain, catheter, tube, etc.) to freely extend from either end of the vertical channel when the housing is engaged with the line (e.g., drain, catheter, tube, etc.). The housing may be adapted to be circumferentially engageable with a line (e.g., drain, catheter, tube, etc.).

In some embodiments, the housing of the securement element comprises a channel that traverses the length of the housing with openings at either end of the channel. In some embodiments, a portion of a medical line secured by a device herein extends into a first opening on the housing, through the interior or central channel, and out the second opening. In some embodiments, the channel is aligned (coaxially) with the vertical axis of the housing. In some embodiments, the channel is centered (e.g., side to side, front to back, etc.) within the housing. In some embodiments, the channel is sized to accommodate a medical line appropriate for the intended use. For example, the channel may be 5-100 Fr in diameter (e.g., 5 Fr, 10 Fr, 15 Fr, 20 Fr, 25 Fr, 30 Fr, 35 Fr, 40 Fr, 45 Fr, 50 Fr, 55 Fr, 60 Fr, 70 Fr, 80 Fr, 90 Fr, 100 Fr, or ranges therebetween (e.g., 10-30 Fr, etc.)). In some embodiments, the channel is 5-20 Fr (e.g., 5 Fr, 6 Fr, 7 Fr, 8 Fr, 9 Fr, 10 Fr, 11 Fr, 12 Fr, 13 Fr, 14 Fr, 14 Fr, 15 Fr, 16 Fr, 17 Fr, 18 Fr, 19 Fr, 20 Fr, or ranges therebetween). In some embodiments, the channel is 1-5 cm in length (e.g., 1 cm, 1.5 cm, 2 cm, 2.5 cm, 3 cm, 3.5 cm, 4 cm, 4.5 cm, 5 cm, or ranges therebetween).

In some embodiments, the securement element comprises a housing. In some embodiments, the housing of the securement element has two sub-components: a rear body and a front body (FIG. 1). In some embodiments, the rear body and the front body each contain a portion (e.g., half) of a lumen where the medical line (e.g., percutaneous drain) is to be placed. Coupling of the rear and front bodies produces a full lumen (i.e., the vertical channel) from the fractional (e.g., half) lumens of the individual bodies. In some embodiments, within one or both fractional (e.g., half) lumens arc small protrusions which allow the device to clamp down and tether on the drain without occluding it. In some embodiments, when positioned within the channel a line is free to slide vertically through the channel. In other embodiments, the housing grips the line within the channel, holding the line in place and thereby preventing the line from sliding vertically through the channel.

In some embodiments, the housing is a clam-shell housing including two bodies that can be coupled together to form the housing. In some embodiments, the front and rear bodies are pivotally coupled to each other. In some embodiments, one or more clips secure the bodies together.

In some embodiments, the front body and the rear body of the device are configured to be coupled together by any suitable mechanism. In some embodiments, one portion of the housing (e.g., the front body) comprises clips on either side that are configured to couple with clip receptacles on the other portion of the housing (e.g., the rear body). In some embodiments, the clips snap into the clip receptacles, thereby coupling bodies together to form a housing (e.g., encompassing the medical line. In other embodiments, the front and rear bodies are coupled on one side by a permanent hinge; a clip and clip receptive on the other side of the bodies allows for the housing and the channel to be fully formed around the medical line. Other components that may find use in securing one or both sides of the front body to the rear body include pins or other protrusions (that fit in corresponding holes), screws (that tighten into screw receptacles, etc.

In some embodiments, extending from the housing of the securement element is a hanger, which is used to couple the securement element to the receiving element. In some embodiments, one or more hangers on the securement element are received by one or more receptacles on the receiving element to securely couple the securement element to the receiving element (e.g., while allowing the securement element to pivot in one plane with respect to the receiving element). In some embodiments, a hanger is a type of fastener capable of securely mating with a receptacle. In some embodiments, a hanger is a rod, pin, bolt, screw, etc. In some embodiments, the shape and size of the hanger is configured to mate with the receptacle to securely couple the securement element to the receiving element. In particular embodiments, the hanger is a rod with an end cap 17 (See FIG. 1B). In some embodiments, the rod is cylindrical. In some embodiments, the end cap is cylindrical or of another suitable cross-sectional shape. In some embodiments, a pair of hangers (e.g., rods with end caps) extend in opposite directions, along a horizontal axis of the housing, and from cither side of the housing. In some embodiments, the housing comprises an opening of the channel at the top of the housing between the two hangers (e.g., rods). In some embodiments, the hangers protrude from the rear body of the housing. In other embodiments, the hangers protrude from the front body of the housing.

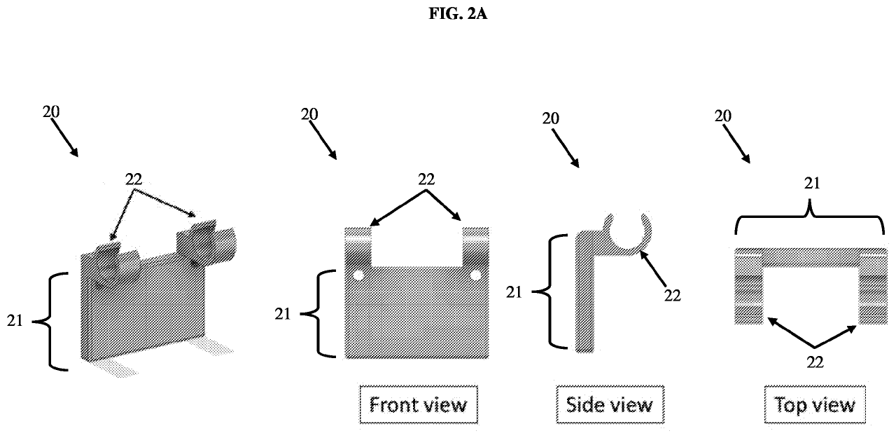

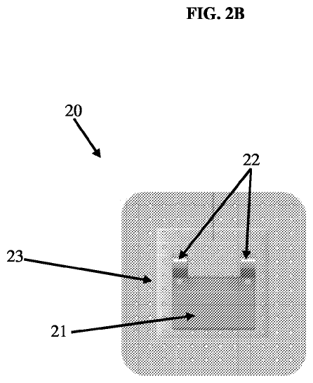

In some embodiments, the devices herein comprise a receiving element (FIG. 2). In some embodiments, the receiving element functions to anchor the device to the subject (e.g., the skin over or adjacent to the point of entry of the medical line into the subject) and couple with the securement element. In some embodiments, a receiving element 20 comprises a mounting plate 21 and receptacles 22 (FIG. 2A). In some embodiments, a receiving element 20 comprises a mounting plate 21 attached to an adhesive element 23 (FIG. 2B).

In some embodiments, the receiving element comprises a surface that can be attached to or adhered to the skin of the subject. In some embodiments, the surface comprises a bandage or dressing. In some embodiments, an adhesive surface of the bandage/dressing is used to adhere the receiving element to the subject. As will be understood, a bandage/dressing backing may comprise a central portion without adhesive, configured to be placed over the point of entry of the medical line into the subject, surrounded by (e.g., a circumferential portion) or flaked by (e.g., edge portions) adhesive for contacting the skin of the subject. In some embodiments, other components of the receiving element are attached to, displayed upon, or coupled to the bandage/dressing.

In some embodiments, the receiving element comprises a mounting plate. In some embodiments, the mounting plate has dimensions on the order of 1-5 cm in length and width (e.g., 1 cm, 1.5 cm, 2 cm, 2.5 cm, 3 cm, 3.5 cm, 4 cm, 4.5 cm, 5 cm, or ranges therebetween). In some embodiments, the mounting plate is attached to or coupled with the bandage/dressing. In such embodiments, securing the bandage/dressing to the subject functions to indirectly secure the mounting plate to the subject. In other embodiments, the mounting plate is directly adhered to the skin of the subject. In such embodiment, the mounting plate may comprise an adhesive surface that allows for direct adhesion.

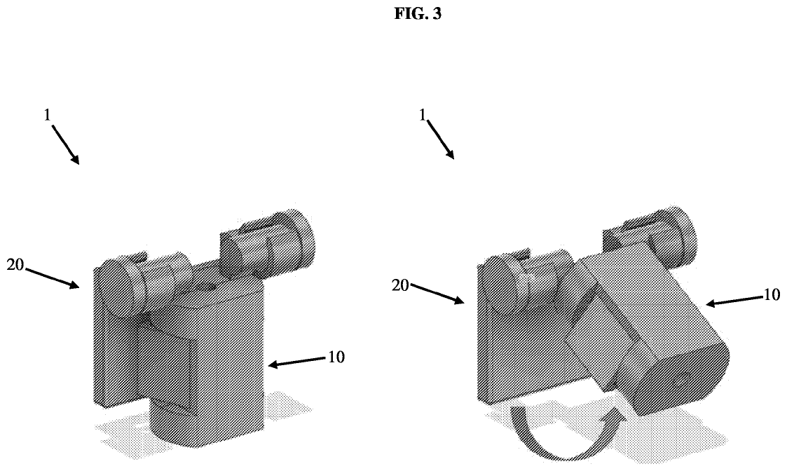

In some embodiments, the receiving element comprises one or more receptacles. The receptacles are configured to receive the hangers of the securement element, thereby coupling the securement element (e.g., with a medical line therethrough) to the receiving element (e.g., secured to the skin surface near the point of entry of the medical line into the subject). In some embodiments, the receptacles comprise partial rings (e.g., 330°, 315°, 300°, 270°, 255°, 240°, 225°, or ranges therebetween or a full ring) that are sized and shaped to receive the hangers. In some embodiments, the hangers “snap” into place within the receptacles. In some embodiments, the receptacles are hooks and the hanger is “hung” therefrom. Other suitable arrangements of hangers and receptacles will be understood. In some embodiments, a device comprises two hangers (e.g., rods) and two receptacles. In some embodiments, the hanger sits within the receptacle in a manner that allows the securement element 10 to pivot in one plane with respect to the receiving element 20 (FIG. 3). In some embodiments, the receptacles extend from the front face of the mounting plate and/or bandage/dressing.

In some embodiments, a device is placed onto the skin of a subject adjacent (e.g., 0.5 cm, 1 cm, 1.5 cm, 2 cm, etc.) to the point of entry of the medical line into the subject. In embodiments in which a bandage/dressing is used to display the receiving element of the device, the bandage/dressing may be placed to cover the point of entry into the subject and position the device adjacent to the point of entry. In such embodiments, an aperture within the bandage allows the medical line to extend from the point of entry, through the bandage/dressing, and into/through the channel of the device. In other embodiments, the bandage/dressing may be placed adjacent to the point of entry.

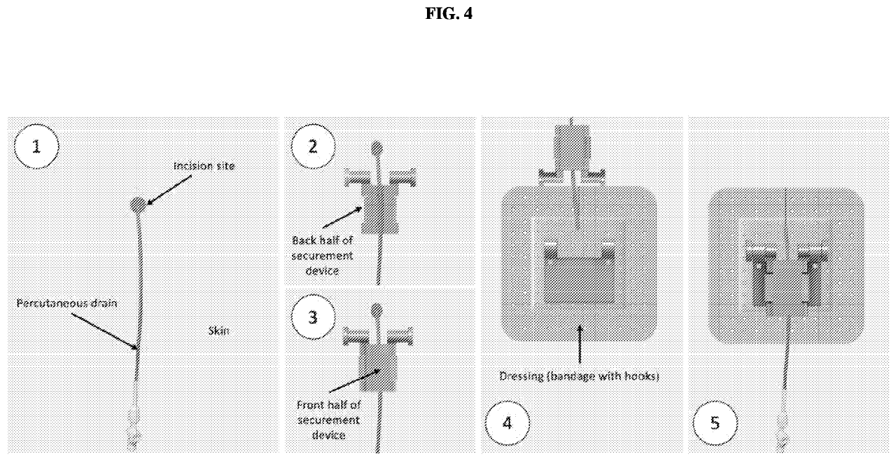

FIG. 4 depicts an exemplary device and five steps for its use in securing a percutaneous drain.

In some embodiments, the devices herein are intended for use with medical lines that are to be used for a period of time of six months or less (e.g., 6 months, 4 months, 2 months, 1 month, 3 weeks, 2 weeks, 1 week, 6 days, 5 days, 4 days, 3 days, 2 days, 1 day, 12 hours, 6 hours, 4 hours, 2 hours, 1 hour, 30 minutes, 10 minutes, or less). In some embodiments, the devices herein are intended for use with medical lines that require frequent, routine scheduled changes.

In some embodiments, devices herein and components there of comprises any suitable materials. Suitable materials include plastics and metals.

The devices, systems, and methods herein provide numerous and significant advantages of existing technologies and methods, as described throughout. For example, the devices herein do not require penetrating the skin to secure the tubing, and therefore are inherently less painful than traditional techniques utilizing sutures. In some embodiments, the devices herein also comprise a joint or hinge that allows the drain to move in one plane, which offers patients more comfort when moving around. Because the devices herein comprise two pieces (e.g., a securement element and receiving element (mounting plate)), in the case of leakage, the bandage can be exchanged while keeping the drain itself in place without migration.

In some embodiments, friction between the device and the tubing (e.g., drain) prevents drain dislodgement. Using traditional securement techniques, excessive movement of an extra-corporeal drain tip leads to loosening of the drain within the body. The pivot/hinge in the devices herein (e.g., between the securement element and receiving element) decreases the movement of the drain within the body and therefore decreases the risk of dislodgement.

1. A line securement device comprising:
(a) a securement element comprising:
(i) a housing defining an interior channel that traverses the securement element a vertical axis of the securement element, wherein the interior channel is configured to allow a medical line to extend through the housing, along the vertical axis, and
(ii) a hanger coupled to the housing and extending along a horizontal axis of the securement element;
(b) a receiving element comprising:
(i) a mounting plate having a front face and a rear face,
(ii) one or more receptacles coupled to the front face of the mounting plate, wherein the one or more receptacles are configured to receive the hanger,
(iii) an adhesive element on the rear face of the mounting plate, wherein the adhesive element is configured to securely couple the mounting plate to a skin of a subject.

2. The device of claim 1, wherein the housing comprises a clam-shell having a rear body and a front body.

3. The device of claim 2, wherein the front body is hingedly coupled to the rear body.

4. The device of claim 2, wherein the housing comprises clips for coupling the front body to the rear body.

5. The device of claim 2, wherein the interior channel resides between the front body and the rear body.

6. The device of claim 2, wherein the hanger is coupled to the rear body of the housing.

7. The device of claim 1, wherein the hanger comprises a rod along the horizontal axis of the securement element.

8. The device of claim 7, wherein the one or more receptacles include a hook-shaped protrusion configured partially circumscribe the cross-sectional shape of the rod.

9. The device of claim 7, wherein the rod is terminated by an end cap of greater circumference than the rod.

10. The device of claim 1, wherein the hanger comprises a pair of rods extending along the horizontal axis of the securement element with a gap between the rods and the one or more receptacles comprise a pair of receptacles configured to respectively receive the pair of rods.

11. The device of claim 1, wherein the receipt of the hanger by the one or more receptacles is configured to provide secure coupling between the securement element and the mounting element.

12. The device of claim 11, wherein the receipt of the hanger by the one or more receptacles is configured to allow the securement element to pivot in a single plane with respect to the mounting element.

13. The device of claim 1, wherein the channel is 5-100 Fr in diameter.

14. The device of claim 1, wherein the adhesive element includes a bandage attached to the rear face of the mounting plate.

15. The device of claim 1, wherein the adhesive element includes a glue applied to the rear face of the mounting plate.

16. A system comprising:
(a) the line securement device of claim 1; and
(b) a medical line coupled to the line securement device.

17. The system of claim 16, wherein the medical line includes one or more selected from the group consisting of a percutaneous drain, a catheter, an endotracheal tube, a tracheotomy tube, chest tube, a vascular catheter, an urinary catheter, a nephrostomy tube, a biliary stent, a cholecystostomy tube, a peritoneal catheter, an epidural catheter, a central nervous system catheter, a prosthetic valve, a gastric tube, an intestinal tube, drug-delivery implant, and an intravenous tube.

18. The system of claim 16, wherein the medical line extends through the channel.

19. The system of claim 16, wherein the securement element is securely coupled to the receiving element by coupling of the hanger and one or more receptacles.

20. A method of securing a medical line to a subject using the line securement device of one of claim 1, the method comprising:
(a) placing the medical line at a first location on the subject;
(b) running the medical line through the channel of the housing of the securement element;
(c) attaching the receiving element to a second location of the subject adjacent to the first location, by the adhesive element;
(d) coupling the securement element to the receiving element by coupling the hanger to the one or more receptacles.

21. The method of claim 20, wherein the placing the medical line at a first location on the subject comprising inserting the medical line into an incision in the subject or an orifice of the subject.

22. The method of claim 20, wherein the medical line includes one or more selected from the group consisting of a percutaneous drain, a catheter, an endotracheal tube, a tracheotomy tube, chest tube, a vascular catheter, an urinary catheter, a nephrostomy tube, a biliary stent, a cholecystostomy tube, a peritoneal catheter, an epidural catheter, a central nervous system catheter, a prosthetic valve, a gastric tube, an intestinal tube, drug-delivery implant, and an intravenous tube.

23. The method of claim 20, wherein the running the medical line through the channel comprises aligning the rear and front bodies of the housing with the medical line and coupling the front body to the rear body, thereby securing the medical line within the channel of the housing.
